(19) United States
(12) Patent Application Publication
(10) Pub. No.: US 2025/0132030 A1
(43) Pub. Date: Apr. 24, 2025

(54) ULTRASONIC DIAGNOSTIC APPARATUS AND ULTRASONIC DIAGNOSTIC METHOD

(71) Applicant: CANON MEDICAL SYSTEMS CORPORATION, Tochigi (JP)

(72) Inventors: Hiroaki ISHIKAWA, Saitama (JP); Seito IGARASHI, Nasushiobara (JP)

(73) Assignee: CANON MEDICAL SYSTEMS CORPORATION, Tochigi (JP)

(21) Appl. No.: 18/924,343

(22) Filed: Oct. 23, 2024

(30) Foreign Application Priority Data
Oct. 24, 2023 (JP) 2023-182809

(51) Int. Cl. G16H 40/63; A61B 8/00; G16H 70/20

(52) U.S. Cl. CPC G16H 40/63; A61B 8/463; G16H 70/20

(57) ABSTRACT

An ultrasonic diagnostic apparatus according to an embodiment includes a processing circuit. The processing circuit is configured to determine which cross section first ultrasonic image data represents, the first ultrasonic image data being image data designated as a reference image for an examination including a process of acquiring an ultrasonic image; to acquire information for enabling a user to acquire the determined cross section; and to store the acquired information in a storage device in association with the first ultrasonic image data.

### CROSS-REFERENCE TO RELATED APPLICATIONS

This application is based upon and claims the benefit of priority from Japanese Patent Application No. 2023-182809, filed on Oct. 24, 2023; the entire contents of which are incorporated herein by reference.

### FIELD

Embodiments described herein relate generally to an ultrasonic diagnostic apparatus and an ultrasonic diagnostic method.

### BACKGROUND

In an ultrasonic diagnostic apparatus, what is called a protocol assistant is known. The protocol assistant provides a function for enabling preregistration of an examination procedure suitable for a target of examination, and a function for displaying the registered examination procedure on a screen and causing the ultrasonic diagnostic apparatus to operate in accordance with the examination procedure.

With such a protocol assistant, users can preregister an examination procedure to be used as a reference in the examination depending on a specific target of examination. By allowing the users to operate the functions of the ultrasonic diagnostic apparatus by following the registered procedure during the examination, erroneous operations of the ultrasonic diagnostic apparatus or mistakes in examination such as a missed step in the examination procedure can be reduced.

Some protocol assistants also have a function for displaying as a reference image a cross-sectional image to be acquired in the next examination procedure. The user adjusts the position or the angle of an ultrasound probe by comparing a current scan image with the reference image.

However, the user still needs to determine to which position or angle the user needs to adjust the ultrasound probe. Therefore, depending on the knowledge level of the user, the user may not be able to make such determinations correctly, and have difficulty in obtaining an intended rendering of the cross-sectional image.

### BRIEF DESCRIPTION OF THE DRAWINGS

FIG. 1 is a schematic illustrating one example of a configuration of an ultrasonic diagnostic apparatus according to an embodiment;

FIG. 2 is a schematic illustrating one example of a display screen of a protocol assistant in the ultrasonic diagnostic apparatus according to a first embodiment;

FIG. 3 is a flowchart illustrating the sequence of a process in the ultrasonic diagnostic apparatus according to the first embodiment;

FIG. 4 is a schematic providing a summary of the process in the ultrasonic diagnostic apparatus according to the first embodiment;

FIG. 5 is a schematic illustrating one example of a reference camera image displayed by the ultrasonic diagnostic apparatus according to the first embodiment;

FIG. 6 is a schematic illustrating one example of a schema displayed by the ultrasonic diagnostic apparatus according to the first embodiment;

FIG. 7 is a schematic illustrating one example of a screen displayed by the ultrasonic diagnostic apparatus according to the first embodiment;

FIG. 8 is a schematic illustrating one example of a screen displayed by the ultrasonic diagnostic apparatus according to the first embodiment;

FIG. 9 is a schematic illustrating one example of a screen displayed by the ultrasonic diagnostic apparatus according to the first embodiment; and

FIG. 10 is a schematic illustrating one example of a screen displayed by an ultrasonic diagnostic apparatus according to a second embodiment.

### DETAILED DESCRIPTION

An ultrasonic diagnostic apparatus according to an embodiment includes a processing circuit. The processing circuit is configured to determine which cross section first ultrasonic image data represents, the first ultrasonic image data being image data designated as a reference image for an examination including a process of acquiring an ultrasonic image; to acquire information for enabling a user to acquire the determined cross section; and to store the acquired information in a storage device in association with the first ultrasonic image data.

### First Embodiment

An ultrasonic diagnostic apparatus and a computer program according to some embodiments will now be explained in detail with reference the drawings.

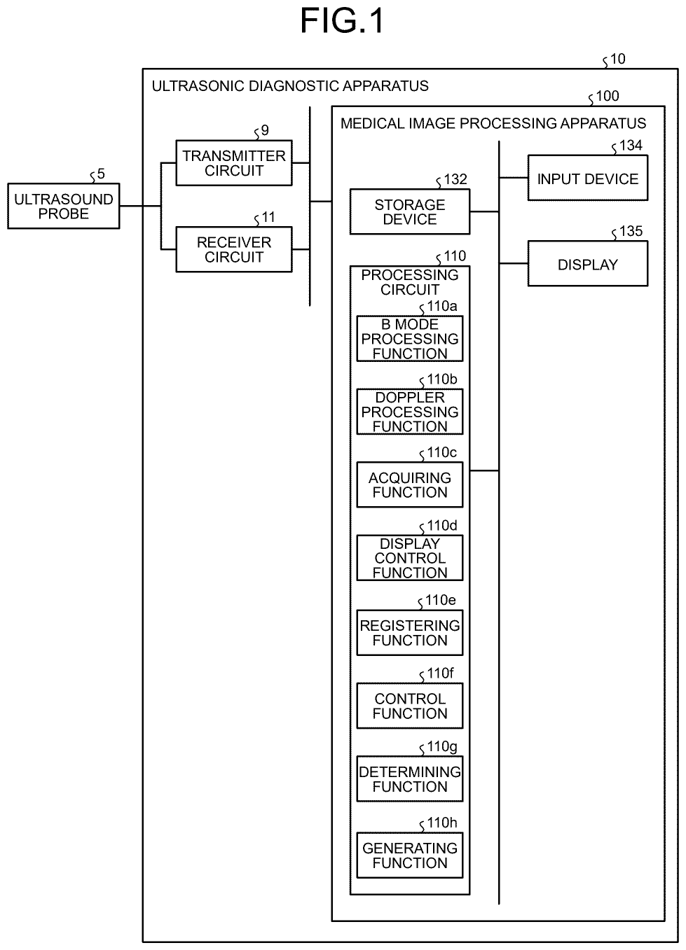

To being with, a configuration of an ultrasonic diagnostic apparatus according to a first embodiment will be explained. FIG. 1 is a block diagram illustrating an exemplary configuration of the ultrasonic diagnostic apparatus according to the first embodiment. As illustrated in FIG. 1, the ultrasonic diagnostic apparatus according to the first embodiment includes an ultrasound probe 5 and an ultrasonic diagnostic apparatus 10. The ultrasonic diagnostic apparatus 10 includes a transmitter circuit 9, a receiver circuit 11, and a medical image processing apparatus 100.

The ultrasound probe 5 includes a plurality of piezoelectric transducer elements, and these piezoelectric transducer elements generate ultrasound on the basis of a driving signal supplied by the transmitter circuit 9, to be described later, included in the ultrasonic diagnostic apparatus 10. The piezoelectric transducer elements included in the ultrasound probe 5 receive a reflection wave from a subject P, and converts the reflection wave into an electric signal (reflection wave signal). The ultrasound probe 5 also includes a matching layer provided to the piezoelectric transducer elements, and a backing material for preventing rearward propagation of the ultrasound from the piezoelectric transducer elements. The ultrasound probe 5 is removably connected to the ultrasonic diagnostic apparatus 10.

When the ultrasound probe 5 transmits ultrasound to the subject P, the transmitted ultrasound is reflected one after another on acoustic impedance discontinuous surfaces in body tissues of the subject P, and received as reflection wave and converted into a reflection wave signal, by the piezoelectric transducer elements in the ultrasound probe 5. The amplitude of the reflection wave signal is determined depending on the difference in the acoustic impedance on the discontinuous surface where the ultrasound is reflected. When the transmitted ultrasonic pulse becomes reflected on a moving blood stream or a surface such as a heart wall, the reflected wave signal goes through a frequency shift due to the Doppler effect, by a degree determined by a velocity component of the moving object, with respect to the direction of ultrasonic transmission.

This embodiment is applicable to a configuration in which the ultrasound probe 5 is a 1D array probe that scans the subject P two-dimensionally, or a mechanical 4D probe or 2D array probe that scans the subject P three-dimensionally.

The ultrasonic diagnostic apparatus 10 is an apparatus for generating ultrasonic image data on the basis of the reflection wave signal received from the ultrasound probe 5. The ultrasonic diagnostic apparatus 10 illustrated in FIG. 1 is an apparatus capable of generating two-dimensional ultrasonic image data on the basis of the two-dimensional reflection wave signal, and also capable of generating three-dimensional ultrasonic image data on the basis of the three-dimensional reflection wave signal. The embodiment is also applicable to a configuration in which the ultrasonic diagnostic apparatus 10 is an apparatus specialized for two-dimensional data.

The ultrasonic diagnostic apparatus 10 includes, as illustrated in FIG. 1, the transmitter circuit 9, the receiver circuit 11, and the medical image processing apparatus 100.

The transmitter circuit 9 and the receiver circuit 11 control transmission and the reception of the ultrasound, performed by the ultrasound probe 5, on the basis of an instruction issued by a processing circuit 110 including a control function 110f, to be described later. The transmitter circuit 9 includes a pulse generator, a transmission delaying unit, and a pulser, and supplies a driving signal to the ultrasound probe 5. The pulse generator repeatedly generates a rate pulse for forming ultrasound to be transmitted, at a predetermined pulse repetition frequency (PRF).

The transmission delaying unit serves to converge the ultrasound generated by the ultrasound probe 5 into a beam, and adds a delay time, for each of the piezoelectric transducer elements, necessary for determining the transmission directivity, to each rate pulse generated by the pulse generator. The pulser applies the driving signal (driving pulse) to the ultrasound probe 5, at the timing based on the rate pulse.

In other words, the transmission delaying unit adjusts the direction for transmitting the ultrasound from the surface of the piezoelectric transducer elements, by changing the delay time added to each of the rate pulses. The transmission delaying unit also controls the position of the convergence point of the ultrasound to be transmitted (the focus of the transmission), in the depth direction, by changing the delay time added to each of the rate pulses.

The transmitter circuit 9 includes, in order to execute a predetermined scanning sequence on the basis of an instruction from the processing circuit 110 to be described later, a function enabled to change a transmission frequency, a transmission driving voltage, and the like, instantaneously. Specifically, a change in the transmission driving voltage is implemented by a linear amplifier oscillation circuit capable of changing the value of the transmission driving voltage instantaneously, or a mechanism for electrically switching a plurality of power source units.

The receiver circuit 11 includes an amplifier circuit, an analog-to-digital (A/D) converter, a reception delaying circuit, an adder, and a quadrature phase detector circuit, and generates a reception signal (reflection wave data) by applying various processes to the reflection wave signal received from the ultrasound probe 5. The amplifier circuit amplifies the reflection wave signal on each channel, and performs gain correction processing. The A/D converter performs an A/D conversion of the reflection wave signal resultant of the gain correction. The reception delaying circuit adds a reception delay time necessary for determining the reception directivity to the digital data. The adder performs adding processing of the reflection wave signals having the reception delay time added by the reception delaying circuit. As a result of this adding processing of the adder, among the reflection components of the reflection wave signal, the reflection component in the direction of the reception directivity is emphasized. The quadrature phase detector circuit then converts the output signal from the adder into an in-phase signal (I signal) and a quadrature-phase signal (Q signal) in the baseband bandwidth. The quadrature phase detector circuit then transmits the I signal and the Q signal (hereinafter referred to as IQ signal) to the processing circuit 110, as reception signals (reflection wave data). The quadrature phase detector circuit may also convert the output signal from the adder into a radio frequency (RF) signal, and transmit the resultant signal to the processing circuit 110. The IQ signal and the RF signal can be said to be the reception signals including phase information.

To scan a two-dimensional region inside the subject P, the transmitter circuit 9 causes the ultrasound probe 5 to transmit an ultrasonic beam for scanning the two-dimensional region. The receiver circuit 11 generates two-dimensional reception signals from two-dimensional reflection wave signals received from the ultrasound probe 5. To scan a three-dimensional region inside the subject P, the transmitter circuit 9 causes the ultrasound probe 5 to transmit an ultrasonic beam for scanning the three-dimensional region. The receiver circuit 11 generates three-dimensional reception signals from the three-dimensional reflection wave signals received from the ultrasound probe 5. The receiver circuit 11 generates reception signals on the basis of the reflection wave signals, and transmits the generated reception signals to the processing circuit 110.

The transmitter circuit 9 transmits an ultrasonic beam from a predetermined transmission position (transmission scan line) with the ultrasound probe 5. The receiver circuit 11 receives a signal resultant of the reflection wave of the ultrasonic beam transmitted from the transmitter circuit 9, at a predetermined reception position (reception scan line) from the ultrasound probe 5. In a setting without the use of simultaneous parallel receptions, the same single scan line is used as the transmission scan line and the reception scan line. In a setting with the use of parallel simultaneous receptions, by contrast, when the transmitter circuit 9 causes the ultrasound probe 5 to transmit a beam of ultrasound from a single transmission scan line once, the receiver circuit 11 causes the ultrasound probe 5 to receive the signal resultant of the reflection wave of the ultrasonic beam having been transmitted from the ultrasound probe 5 caused by the transmitter circuit 9, at a plurality of predetermined reception positions (reception scan lines) at the same time, as a plurality of reception beams.

The medical image processing apparatus 100 is connected to the transmitter circuit 9 and the receiver circuit 11, and executes control of processing of the signals received from the receiver circuit 11, and control of the transmitter circuit 9. The medical image processing apparatus 100 includes the processing circuit 110, a storage device 132, an input device 134, and a display 135. The processing circuit 110 includes a B mode processing function 110a, a Doppler processing function 110b, an acquiring function 110c, a display control function 110d, a registering function 110e, the control function 110f, a determining function 110g, and a generating function 110h.

In this embodiment, a trained model and the processing functions executed by the B mode processing function 110a, the Doppler processing function 110b, the acquiring function 110c, the display control function 110d, the registering function 110e, the control function 110f, the determining function 110g, and the generating function 110h are stored in the storage device 132, as computer-executable programs. The processing circuit 110 is a processor for implementing the function corresponding to each of the computer programs, by reading the computer program from the storage device 132, and by executing the computer program. In other words, the processing circuit 110 having read the computer programs has the functions indicated inside the processing circuit 110 in FIG. 1. Illustrated in FIG. 1 is an example in which the functions of the processing circuit 110 are implemented by a single processing circuit, but the processing circuit 110 may also be provided as a combination of a plurality of independent processors, and be configured to implement the functions by causing each of the processors to execute a computer program. In other words, the functions listed above may be provided as computer programs, and a single processing circuit may execute each one of the computer programs. Furthermore, a single processing circuit may implement two or more functions, among the functions provided to the processing circuit 110. As another example, a specific function may be implemented on a dedicated independent circuit for executing a computer program.

The B mode processing function 110a, the Doppler processing function 110b, the acquiring function 110c, the display control function 110d, the registering function 110e, the control function 110f, the determining function 110g, and the generating function 110h illustrated in FIG. 1 are examples of a B mode processing unit, a Doppler processing unit, an acquiring unit, a display control unit, a registering unit, a control unit, and a determining unit.

The term “processor” used in the above description includes, for example, a central processing unit (CPU), a graphical processing unit (GPU), or a circuit such as an application-specific integrated circuit (ASIC) or a programmable logic device (such as a simple programmable logic device (SPLD), a complex programmable logic device (CPLD), and a field-programmable gate array (FPGA)). Such a processor implements a function by reading a computer program stored in the storage device 132 and executing the computer program.

The computer program may also be incorporated directly into the circuit of the processor, instead of storing the computer program in the storage device 132. In such a configuration, the processor implements a function by reading the computer program incorporated in the circuit, and executing the computer program. Furthermore, the transmitter circuit 9, the receiver circuit 11, and the like included in the ultrasonic diagnostic apparatus 10 are sometimes implemented as hardware, such as an integrated circuit, but sometimes as computer programs that are implemented as software modules.

The processing circuit 110 is a processing unit that performs various types of signal processing to reception signals received from the receiver circuit 11. The processing circuit 110 includes the B mode processing function 110a, the Doppler processing function 110b, the acquiring function 110c, the display control function 110d, the registering function 110e, the control function 110f, and the determining function 110g.

The acquiring function 110c, the registering function 110e, and the determining function 110h will be described later in detail.

With the B mode processing function 110a, the processing circuit 110 receives data from the receiver circuit 11, and applies processing such as logarithmic amplification, envelope detection, and logarithmic compression thereto, to generate data in which the signal intensity is represented as brightness (B mode data).

With the Doppler processing function 110b, the processing circuit 110 makes a frequency analysis of velocity information from the reception signals (reflection wave data) received from the receiver circuit 11, and generates data that is extraction of information such as velocities, variance, and power of moving bodies, as having been affected by the Doppler effect, for a plurality of points (Doppler data).

The B mode processing function 110a and the Doppler processing function 110b illustrated in FIG. 1 are capable of processing two-dimensional reflection wave data as well as three-dimensional reflection wave data. With the display control function 110d, the processing circuit 110 controls the display 135 to display an ultrasonic image data to be displayed, the image data being stored in the storage device 132.

With the control function 110f, the processing circuit 110 controls the entire processing of the ultrasonic diagnostic apparatus. Specifically, with the control function 110f, the processing circuit 110 controls the processing of the transmitter circuit 9, the receiver circuit 11, and the processing circuit 110 on the basis of various setting requests entered by an operator via the input device 134, or various control programs and various types of data read from the storage device 132.

With the generating function 110h, the processing circuit 110 generates ultrasonic image data from the data generated by the B mode processing function 110a and the Doppler processing function 110b. Also with the generating function 110h, the processing circuit 110 generates two-dimensional B mode image data, in which intensities of reflection waves are represented as brightness, from the two-dimensional B mode data generated by the B mode processing function 110a. Also with the generating function 110h, the processing circuit 110 generates two-dimensional Doppler image data representing information of moving bodies, from the two-dimensional Doppler data generated by the Doppler processing function 110b. The two-dimensional Doppler image data is velocity image data, variance image data, or power image data, or image data that is a combination thereof.

Also with the generating function 110h, the processing circuit 110 converts (scan-converts) a scan-line signal sequence resultant of an ultrasonic scan to a scan-line signal sequence in a video format, a representative example of which is that for a television, to generate ultrasonic image data for display. Also with the generating function 110h, the processing circuit 110 performs, as some examples of various types of image processing other than the scan conversion, image processing for reproducing an average-brightness image using a plurality of image frames after the scan conversion (smoothing processing), or image processing using a differential filter across an image (edge emphasizing processing). Also with the generating function 110h, the processing circuit 110 performs various types of rendering processing to volume data, to generate two-dimensional image data that is the volume data to be displayed on the display 135.

The storage device 132 is implemented as a semiconductor memory element such as a random-access memory (RAM) and a flash memory, a hard disk, or an optical disc, for example. The storage device 132 is a memory for storing therein data such as the image data to be displayed, the image data being generated by the processing circuit 110. The storage device 132 may also store therein data generated by the B mode processing function 110a or the Doppler processing function 110b. An operator can call the B mode data or the Doppler data stored in the storage device 132 after the diagnosis, for example, and the data is turned into ultrasonic image data for display, through the processing circuit 110. The storage device 132 may also store therein the reception signals (reflection wave data) output from the receiver circuit 11.

The storage device 132 also stores therein control programs for transmitting and receiving ultrasound, image processing, and displaying processing, diagnosis information (e.g., patient ID, opinions made by physicians), and various types of data such as diagnosis protocols and various body marks, as required.

The input device 134 receives inputs of various instructions or information from the operator. Examples of the input device 134 includes a pointing device such as a mouse or a trackball, a selection device such as a mode select switch, or an input device such as a keyboard.

The display 135 displays a graphical user interface (GUI) for receiving inputs of imaging conditions, or images generated by the generating function 110h or the like, for example, under the control of the control function 110f or the like. The display 135 is a display device such as a liquid crystal display. The display 135 is an example of a display unit. The display 135 is provided with a mouse, a keyboard, a button, a panel switch, a touch command screen, a foot switch, a trackball, and a joystick, for example.

The background of the embodiment will now be explained.

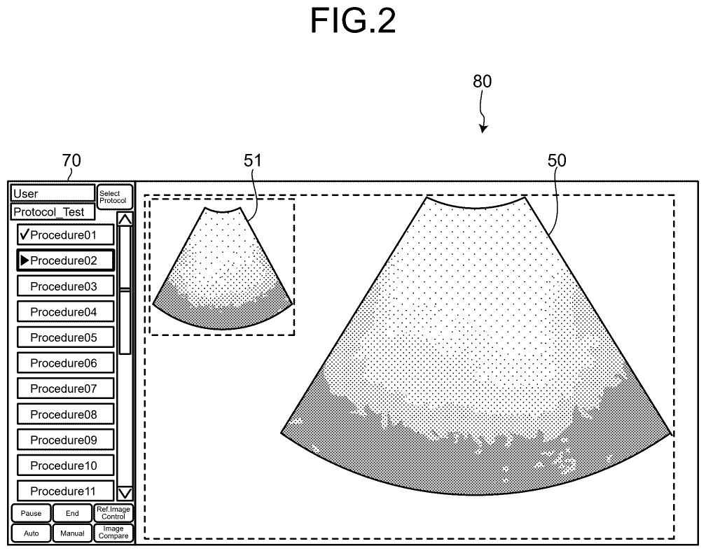

In an ultrasonic diagnostic apparatus, what is called a protocol assistant is known. A protocol assistant provides a function for enabling preregistration of an examination procedure suitable for a target of examination, and a function for displaying the registered examination procedure on the screen and causing the ultrasonic diagnostic apparatus to operate in accordance with the examination procedure. FIG. 2 illustrates one example of a display screen of the protocol assistant for assisting setting of an ultrasonic diagnosis.

As one example, the display screen displayed by such a protocol assistant 80 includes a setting user interface 70, an image 50 that is an ultrasonic image, and a reference image 51 that is a reference ultrasonic image. The image 50 is an ultrasonic image from actual diagnostic imaging executed on the basis of the setting of the protocol assistant 80, and the reference image 51 is a reference ultrasonic image set by the protocol assistant 80 for the use as a reference when a user executes the actual diagnostic imaging. The setting user interface 70 is a setting user interface for performing settings including the designation of the reference ultrasonic image and the like.

With such a protocol assistant, the user can preregister an examination procedure to be used as a reference in the examination of a specific target. By allowing the user to operate the functions of the ultrasonic diagnostic apparatus 10 by following the registered procedure during the examination, erroneous operations of the ultrasonic diagnostic apparatus 10 or mistakes in examination such as a missed step in the examination procedure can be reduced. The ultrasonic diagnostic apparatus 10 is generally a user-dependent image diagnostic apparatus, with results obtained therefrom tending to vary largely; however, with the use of a protocol assistant, because users conduct examinations based on a certain examination criteria presented by the protocol assistant function, such user-dependent variations in the results are less likely to occur.

Some protocol assistants also have a function for displaying as a reference image a cross-sectional image to be acquired in the next examination procedure. The user adjusts the position or the angle of the ultrasound probe by comparing a current scan image with the reference image.

However, the user still needs to determine to which position or angle the user needs to adjust the ultrasound probe. Because anatomical knowledge is required to make such determinations, depending on the knowledge level of the user, the user may not be able to make such determinations correctly, and have difficulty in obtaining an intended rendering of the cross-sectional image.

The embodiment has such a background as its basis, and the ultrasonic diagnostic apparatus 10 according to the embodiment includes the processing circuit 110. The processing circuit 110 is configured to cause the determining function 110g to determine which cross section first ultrasonic image data represents, the first ultrasonic image data being image data designated as a reference image for an examination including a process of acquiring an ultrasonic image. The processing circuit 110 is also configured to cause the acquiring function 110c to acquire information for enabling a user to acquire the determined cross section, and to store the acquired information in the storage device 132 in association with the first ultrasonic image data.

In the ultrasonic diagnostic apparatus 10 according to the embodiment, the processing circuit 110 includes the display control function 110d. The processing circuit 110 is configured to, upon receiving an execution of the examination procedures for an examination including the process of acquiring a registered ultrasonic image, cause the display control function 110d to display the examination procedures as a list in the order of execution, on the display 135 as the display unit. The processing circuit 110 is also configured to, when an examination procedure is to be executed, cause the display control function 110d to acquire information associated with the first ultrasonic image data, the information being information for enabling the user to acquire the cross section of the first ultrasonic image data designated as a reference image, from the storage device 132, and to display the information on the display 135 as the display unit.

Furthermore, a computer program according to the embodiment is configured to cause a computer to execute processing including: determining which cross section first ultrasonic image data represents, the first ultrasonic image data being image data designated as a reference image for an examination including a process of acquiring an ultrasonic image; acquiring information for enabling a user to acquire the determined cross section; and storing the acquired information in a storage device in association with the first ultrasonic image data.

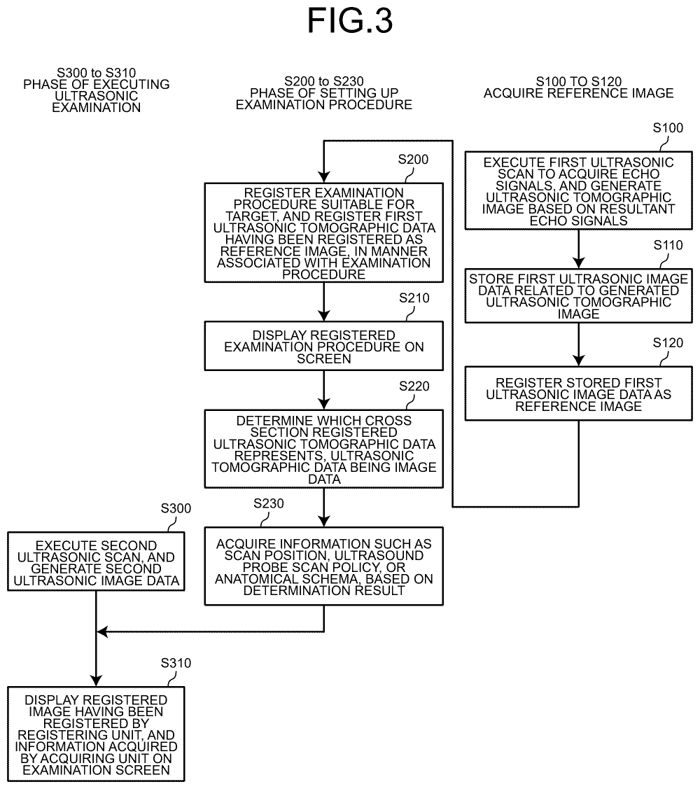

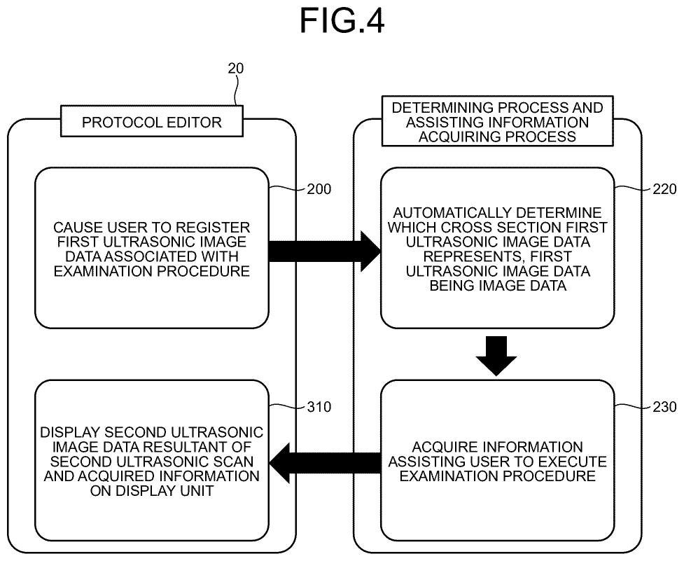

Such configurations will now be explained with reference to FIG. 3, while referring to FIGS. 4 to 9 as appropriate. FIG. 3 is a schematic illustrating the sequence of a process executed by the ultrasonic diagnostic apparatus 10 according to the first embodiment. FIG. 4 is a schematic providing a summary of the process executed by the ultrasonic diagnostic apparatus according to the first embodiment.

In FIG. 3, Steps S200 to S230 represent a process in a phase of setting up an ultrasonic examination procedure for a subject; Steps S100 to S120 represent a process for acquiring a reference image to be used during the setting phase; and Steps S300 to S310 represent a process in a phase of executing the ultrasonic examination of the subject.

To begin with, the process for acquiring a reference image will be explained. At Step S100, the ultrasonic diagnostic apparatus 10 executes a first ultrasonic scan in which the ultrasound probe 5 scans a cross section of a subject with ultrasound, using the transmitter circuit 9 and the receiver circuit 11, and acquires echo signals. The processing circuit 110 then causes the acquiring function 110c to acquire the resultant echo signals. The processing circuit 110 then causes the generating function 110h to generate an ultrasonic tomographic image, on the basis of the acquired echo signals.

At Step S110, the processing circuit 110 transmits first ultrasonic image data related to the ultrasonic tomographic image generated at Step S100 to the storage device 132. The storage device 132 stores therein the received first ultrasonic image data. The first ultrasonic image data herein is ultrasonic tomographic data providing the basis for generating of a reference image, which will be described later.

At Step S120, the processing circuit 110 causes the registering function 110e to register information of the stored first ultrasonic image data, in a manner associated with information of the scan having been carried out.

The process in the phase of setting up an ultrasonic examination procedure for a subject will now be explained. As one example, the ultrasonic diagnostic apparatus 10 causes the protocol assistant 80 for assisting setup of an ultrasonic diagnosis, to set up an ultrasonic examination procedure for a subject. The protocol assistant 80 is implemented using the various functions of the processing circuit 110, including the acquiring function 110c, the display control function 110d, the registering function 110e, the determining function 110g, and the generating function 110h. Typically, a display screen displayed by the protocol assistant 80 includes, for example, the setting user interface 70, the image 50 that is an ultrasonic image, and the reference image 51 that is a reference ultrasonic image, as described above. The image 50 herein is an ultrasonic image resultant of actual diagnostic imaging executed on the basis of the setting of the protocol assistant 80, and the reference image 51 is a reference ultrasonic image to be used as a reference by a user, when the user executes the actual diagnostic imaging, the reference ultrasonic image being designated by the protocol assistant 80. The setting user interface 70 is a setting user interface for performing settings including the designation of the reference ultrasonic image.

At Step S200, before executing a second ultrasonic scan that is the actual diagnostic imaging of the subject in the ultrasonic examination, the user registers an examination procedure suitable for the target, using the setting user interface 70. For example, as illustrated in FIG. 4, the user registers the first ultrasonic image data associated with the examination procedure, using the setting user interface 70 serving as a protocol editor 20. The first ultrasonic image data herein is ultrasonic image data designated as a reference image for the examination including a process of acquiring an ultrasonic image.

Specifically, the processing circuit 110 causes the display control function 110d and the registering function 110e to receive an examination procedure suitable for the target from the user, via the setting user interface 70. As one example, the processing circuit 110 receives a designation of what the target region is, from the user, via the display control function 110d. The processing circuit 110 having received the input of the target then causes the display control function 110d to display a plurality of candidate imaging protocols related to the target on the display screen, and receives a selection of the imaging protocol from the user. The processing circuit 110 then causes the registering function 110e to determine an examination procedure on the basis of the selected imaging protocol, and to register the determined examination procedure as the examination procedure that is suitable for the target. The processing circuit 110 also causes the registering function 110e to acquire the first ultrasonic image data associated with the examination procedure and designated as the reference image, on the basis of the association between the first ultrasonic scan and the ultrasonic image data, registered at Step S120.

At Step S210, the processing circuit 110 causes the display control function 110d to display the examination procedure registered at Step S200 on the display 135, and causes the ultrasonic diagnostic apparatus 10 to operate in accordance with the determined examination procedure.

At Step S220, the processing circuit 110 causes the determining function 110g to determine which cross-section the first ultrasonic tomographic data represents, the first ultrasonic image being image data registered at Step S200 and designated as a reference image, as indicated as a block 220 in FIG. 4. As one example of a method for automatically determining the cross section, the processing circuit 110 causes the determining function 110g to determine the cross section on the basis of a trained model having been trained using machine learning with labeled data. As one example, the processing circuit 110 causes the determining function 110g to compare the features of an input image with those of labeled cross section data having been already learned, and gives the input image with a label given to a piece of cross section data having similar features as those of the input image.

At Step S230, the processing circuit 110 causes the acquiring function 110c to acquire the information for enabling the user to acquire the cross section having been determined by the determining function 110g at Step S220 and to store the acquired information in the storage device 132 in association with the first ultrasonic image data. As one example, as indicated in a box 230 in FIG. 4, the processing circuit 110 causes the acquiring function 110c to acquire the information for enabling the user to acquire the cross section determined by the determining function 110g at Step S220, as information assisting the user to execute the examination procedure. The information for enabling the user to acquire the cross section herein includes, for example, at least one of the scan position for acquiring the cross section, an ultrasound probe scan policy, and an anatomical schema.

One example of the information including the scan position for acquiring the cross section is information indicating that the probe is used to scan at a given position of an apical region of the heart. As another example, the scan position for acquiring the cross section is information indicating that scanning is performed by moving the probe to the apical region of the heart, and then tilting the ultrasonic beam toward the cardiac base extensively. The processing circuit 110 causes the acquiring function 110c to acquire such information on the basis of the position of the cross section acquired at Step S220, for example.

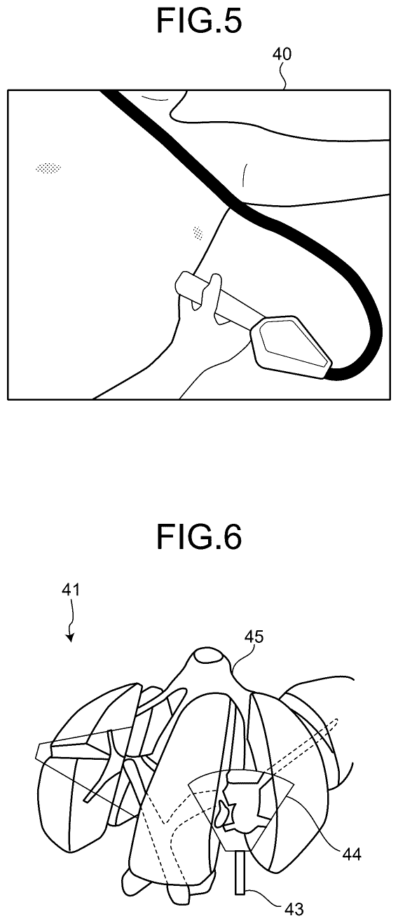

An example of the information including the ultrasound probe scan policy includes an image 40 visually presenting a desirable scan position and scan angle, as illustrated in FIG. 5. The processing circuit 110 causes the acquiring function 110c to generate such an image 40 on the basis of the position of the cross section acquired at Step S220.

One example of how the image 40 is generated will now be explained. Referring back to Step S100, the processing circuit 110 causes the acquiring function 110c to acquire first position information that is information of a relative position of the ultrasound probe 5 and the subject at the time when the first ultrasonic image data is acquired. As one example, the processing circuit 110 causes the acquiring function 110c to acquire a first camera image resultant of capturing an image of the position of the ultrasound probe 5 and the subject at the time when the first ultrasonic image data is acquired. At Step S230, the processing circuit 110 causes the acquiring function 110c to generate the first camera image as the image 40. In this manner, at Step S230, the processing circuit 110 causes the acquiring function 110c to acquire information for enabling the user to acquire a cross section, using the first camera image of the position of the ultrasound probe 5 and the subject at the time when the first ultrasonic image data is acquired. In the manner described above, the processing circuit 110 can acquire the first camera image that is the first position information of a relative position of the ultrasound probe 5 and the subject at the time when the first ultrasonic image data is acquired, using the acquiring function 110c, as the information for enabling the user to acquire the cross section.

FIG. 6 illustrates an example of an anatomical schema 41. The anatomical schema 41 in FIG. 6 is a schema in which a position 43 of the ultrasound probe 5 and a scanned range 44 are superimposed over a diagram of a heart 45. The processing circuit 110 causes the acquiring function 110c to generate the anatomical schema 41 on the basis of the position of the cross section acquired at Step S220.

One example of how the anatomical schema 41 is generated will now be explained. Referring back to Step S100, the processing circuit 110 causes the acquiring function 110c to acquire the first position information that is information of a relative position of the ultrasound probe 5 and the subject at the time when the first ultrasonic image data is acquired. As one example, the processing circuit 110 causes the acquiring function 110c to acquire the information of the position or the angle of the ultrasound probe 5, from a position sensor, a pressure sensor, a camera image, or the like at the time when the first ultrasonic scan for acquiring the first ultrasonic image data is executed.

At Step S120, the processing circuit 110 causes the registering function 110e to register the information of the position or the angle of the ultrasound probe 5 at the time of the first ultrasonic scan, in association with the first ultrasonic image data that is the data of the reference image.

At Step S230, the processing circuit 110 causes the acquiring function 110c to generate the anatomical schema 41 that is the information for enabling the user to acquire the cross section determined at Step S220, using the information obtained from the position sensor or the pressure sensor, or a camera image, at the time when the first ultrasonic scan for acquiring the first ultrasonic image data is executed at Step S100.

Step S300 and Step S310 included as the process in the phase of executing the ultrasonic examination will now be explained. At Step S300, the ultrasonic diagnostic apparatus 10 starts an ultrasonic scan that is the actual diagnostic imaging of the subject, as the second ultrasonic scan. Specifically, the ultrasonic diagnostic apparatus 10 causes the ultrasound probe 5 to scan a cross section of the subject with ultrasound, using the transmitter circuit 9 and the receiver circuit 11, and acquire echo signals for the actual diagnostic imaging. The processing circuit 110 then causes the acquiring function 110c to acquire the resultant echo signals. The processing circuit 110 causes the generating function 110h to generate second ultrasonic image data on the basis of the acquired echo signals. Upon receiving an execution of the examination procedures for an examination including a process of acquiring a registered ultrasonic image, the processing circuit 110 causes the display control function 110d to display the examination procedures as a list in the order of execution, on the display 135 as the display unit.

At Step S310, the processing circuit 110 causes the display control function 110d to display the image registered by the registering function 110e at Step S200, and the information acquired by the acquiring function 110c at Step S230, on the examination screen. In other words, as indicated in a box 310 in FIG. 4, the processing circuit 110 causes the display control function 110d to display the second ultrasonic image data acquired by the second ultrasonic scan, and the information for enabling the user to acquire the cross section determined at Step S220 (that is, the information assisting the user to execute the examination procedure, acquired at Step S230), on the display 135 as the display unit.

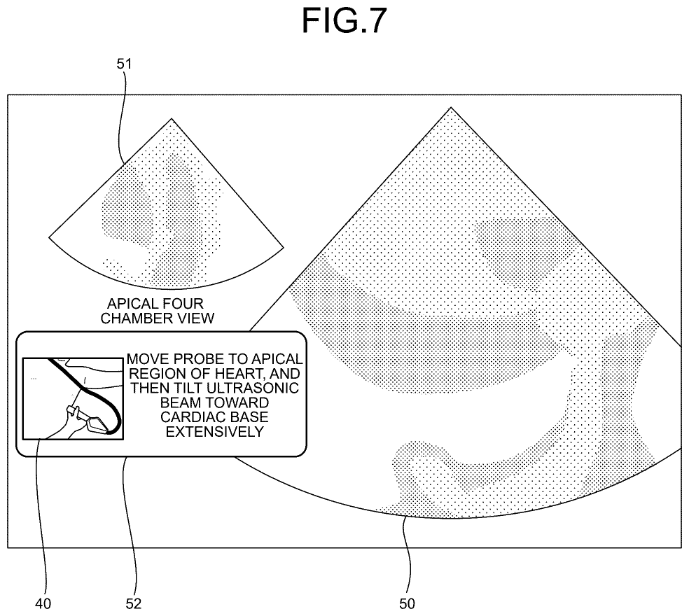

An example of such a process is illustrated in FIGS. 7 to 9. FIG. 7 illustrates one example of a display screen displayed on the display 135 at Step S310, by the processing circuit 110 using the display control function 110d, when the information for enabling the user to acquire the cross section determined at Step S220 and acquired at Step S230 is the image 40 visually presenting a desirable scan position and angle of the ultrasound probe 5 for a target examination.

Upon receiving an execution of the registered examination at Step S300, the processing circuit 110 causes the display control function 110d to display a plurality of examination procedures as a list in the order of execution, on the display 135 as the display unit, at Step S310. The processing circuit 110 causes the display control function 110d to display the second ultrasonic image data acquired by the second scan that is the actual diagnostic imaging, as the image 50, on the display 135. In other words, the processing circuit 110 causes the display control function 110d to display the second ultrasonic image data acquired during the execution of the examination procedure, on the display 135 as the display unit.

The processing circuit 110 causes the display control function 110d to display the reference image registered at Step S200 on the display 135, as the reference image 51. The processing circuit 110 also causes the display control function 110d to display the information for enabling the user to acquire the cross section determined at Step S220 in a display area 52, that is, to display the information assisting the user to execute the examination procedure, acquired at Step S230.

In other words, the processing circuit 110 causes the display control function 110d to acquire the information for enabling the user to acquire the cross section determined at Step S220 from the storage device 132, the information being associated with the first ultrasonic image data, and to display the information on the display 135 as the display unit, when the examination procedure is to be executed. Specifically, the processing circuit 110 causes the display control function 110d to display the image 40 visually presenting a desirable scan position and angle of the ultrasound probe 5 for a target examination, in the display area 52. The processing circuit 110 also causes the display control function 110d to display information to “move the probe to the apical region of the heart, and then tilt the ultrasonic beam toward the cardiac base extensively” that is the information giving a user an instruction related to a desirable way to operate the ultrasound probe 5 to achieve the desirable scan position and angle of the ultrasound probe 5, in the display area 52.

The display area 52 may be presented in any size at any coordinates.

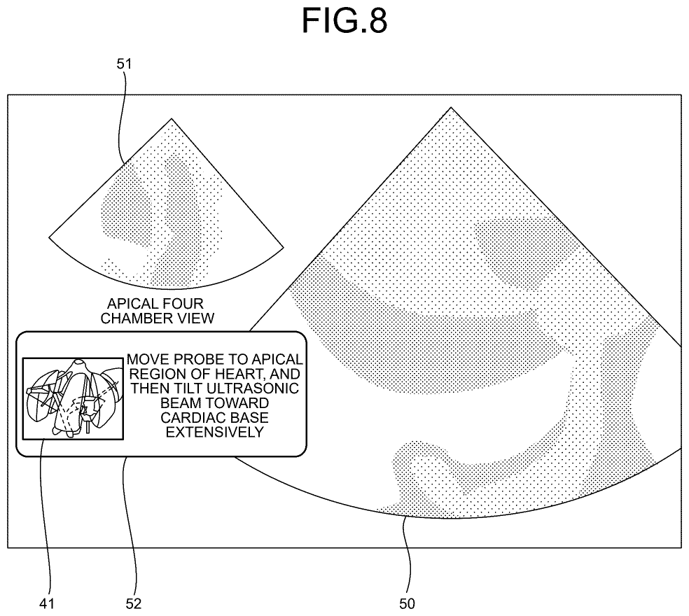

FIG. 8 illustrates one example of the display screen displayed on the display 135 at Step S310, by the processing circuit 110 using the display control function 110d, when the information for enabling the user to acquire the cross section determined at Step S220 and acquired at Step S230 is the anatomical schema 41 illustrated in FIG. 6.

Upon receiving an execution of the registered examination at Step S300, the processing circuit 110 causes the display control function 110d to display a plurality of examination procedures as a list in the order of execution, on the display 135 as the display unit, at Step S310. The processing circuit 110 causes the display control function 110d to display the second ultrasonic image data acquired by the second scan that is the actual diagnostic imaging, as the image 50, on the display 135. In other words, the processing circuit 110 causes the display control function 110d to display the second ultrasonic image data acquired during the execution of the examination procedure, on the display 135 as the display unit.

The processing circuit 110 causes the display control function 110d to display the reference image registered at Step S200 on the display 135, as the reference image 51. The processing circuit 110 also causes the display control function 110d to display the information for enabling the user to acquire the cross section determined at Step S220 in a display area 52, that is, to display the information assisting the user to execute the examination procedure, acquired at Step S230.

In other words, the processing circuit 110 causes the display control function 110d to acquire the information for enabling the user to acquire the cross section determined at Step S220 from the storage device 132, the information being associated with the first ultrasonic image data, and to display the information on the display 135 as the display unit, when the examination procedure is to be executed. Specifically, the processing circuit 110 causes the display control function 110d to display the anatomical schema 41 in which an icon of the ultrasound probe is superimposed over a schema of the target region, at an appropriate position of the ultrasound probe 5, in the display area 52. The processing circuit 110 also causes the display control function 110d to display information “to move the probe to the apical region of the heart, and then tilt the ultrasonic beam toward the cardiac base extensively” that is the information giving a user an instruction related to a desirable way to operate the ultrasound probe 5 to achieve the desirable scan position and angle of the ultrasound probe 5, in the display area 52.

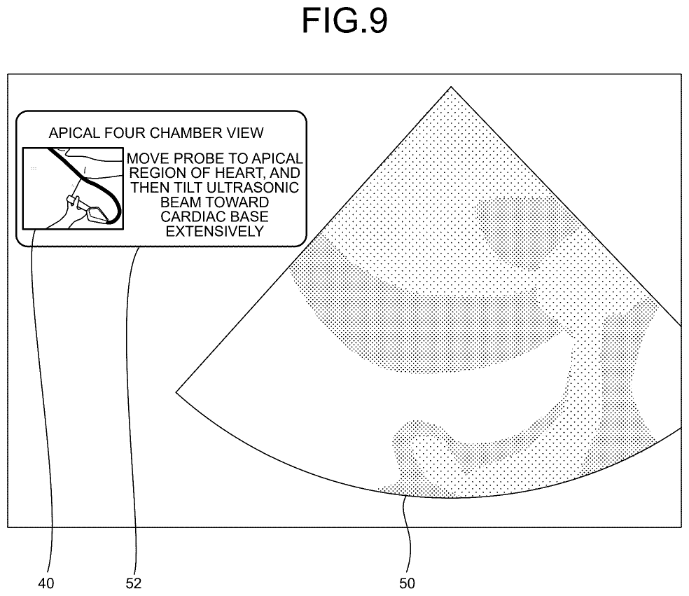

Another example of the user interface is illustrated in FIG. 9. FIG. 9 illustrates the same user interface as that illustrated in FIG. 7 but displaying of the reference image is omitted.

Upon receiving an execution of the registered examination at Step S300, the processing circuit 110 causes the display control function 110d to display a plurality of examination procedures as a list in the order of execution, on the display 135 as the display unit, at Step S310. The processing circuit 110 causes the display control function 110d to display the second ultrasonic image data acquired by the second scan that is the actual diagnostic imaging, as the image 50, on the display 135.

The processing circuit 110 does not cause the display control function 110d to display the reference image registered at Step S200 on the display 135, as the reference image 51.

The processing circuit 110 causes the display control function 110d to acquire the information for enabling the user to acquire the cross section determined at Step S220 from the storage device 132, the information being associated with the first ultrasonic image data, and to display the information on the display 135 as the display unit, when the examination procedure is to be executed. Specifically, the processing circuit 110 causes the display control function 110d to display the image 40 visually presenting a desirable scan position and angle of the ultrasound probe 5 for a target examination, in the display area 52. The processing circuit 110 also causes the display control function 110d to display information to “move the probe to the apical region of the heart, and then tilt the ultrasonic beam toward the cardiac base extensively” that is the information giving a user an instruction related to a desirable way to operate the ultrasound probe 5 to achieve the desirable scan position and angle of the ultrasound probe 5, in the display area 52.

As described above, in the first embodiment, the ultrasonic diagnostic apparatus 10 determines which cross-section the registered ultrasonic image data represents, the ultrasonic image data being the image data, and acquires information for enabling the user to acquire the determined cross section. With this, even a user with a level of knowledge that is not so advanced can achieve better rendering of a desired cross section. Furthermore, because the function for automatically determining the cross section of the reference image and automatically assigning information is provided, person-hours of an imaging protocol creator who is a user is reduced, and usability is improved.

### Second Embodiment

The embodiment is not limited to the example described above. In a second embodiment, an example in which the processing circuit 110 causes the acquiring function 110c to acquire position information of the like of the ultrasound probe 5 during the execution of the second ultrasonic scan at Step S300, executing processing on the basis of the acquired information, will be explained.

Specifically, at Step S300, the processing circuit 110 causes the acquiring function 110c to acquire second position information that is information of a relative position of the ultrasound probe 5 and the subject while the second ultrasonic image data is being collected. Specifically, the processing circuit 110 causes the acquiring function 110c to acquire the position information or the angle information of the ultrasound probe 5 from the position sensor, the pressure sensor, or a camera image, for example, while the second ultrasonic scan is being executed. At Step S310, the processing circuit 110 then causes the generating function 110h to generate guide information for guiding how the position and the angle of the ultrasound probe 5 are to be adjusted, on the basis of the difference between the position information or the like of the ultrasound probe 5 acquired during the collection of the second ultrasonic image data at Step S300, and the position information or the like of the ultrasound probe 5 in the reference image registered at Step S120. As one example, the processing circuit 110 causes the generating function 110h to generate guide information indicating that it is desirable to move the ultrasound probe 5 in a predetermined direction, on the basis of the position information or the like of the ultrasound probe 5 during the collection of the second ultrasonic image data and the position information or the like of the ultrasound probe 5 during the collection of the first ultrasonic image data. The processing circuit 110 then causes the display control function 110d to display the generated guide information on the display 135.

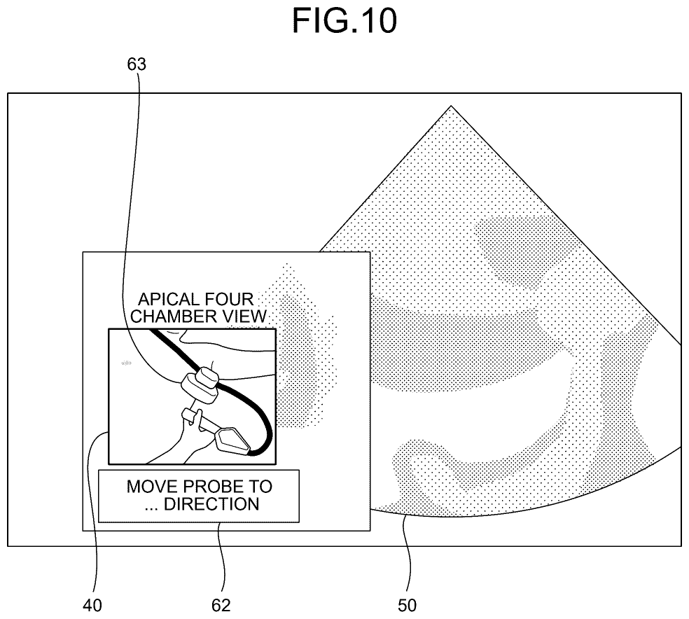

FIG. 10 illustrates an example of such a process. At Step S310, the processing circuit 110 causes the display control function 110d to display the second ultrasonic image data on the display 135, as the image 50, on the basis of the second position information. The processing circuit 110 also causes the display control function 110d to display the image 40 visually presenting the desirable scan position and angle acquired at Step S230 on the display 135. The processing circuit 110 also causes the display control function 110d to display guide information resultant of comparing the position information or the like of the ultrasound probe 5 during the collection of the second ultrasonic image data with the position information or the like of the ultrasound probe 5 during the collection of the first ultrasonic image data, in a display area 62.

The processing circuit 110 may also cause the display control function 110d to display an icon representing the current position and orientation of the ultrasound probe 5, in a manner superimposed over the first camera image, on the basis of the second position information. For example, as illustrated in FIG. 10, the processing circuit 110 may cause the display control function 110d to display an icon 63 representing the current position and orientation of the ultrasound probe 5 in a manner superimposed over the image 40 acquired from the first camera image, on the basis of the second position information. In the manner described above, by displaying the icon 63 of the ultrasound probe 5 over the image 40 that is a camera image at the time at which the reference image is acquired, on the basis of the current position and angle information of the ultrasound probe 5, and matching the position of the icon 63 to the probe position in the image 40 at the time when the reference image is acquired, the processing circuit 110 can guide the user so as to enable the user to acquire the cross section.

As described above, in the second embodiment, the position, the angle information, and the like of the ultrasound probe 5 is collected while the second ultrasonic scan is executed, and the guide information is automatically generated on the basis of the position, the angle information, and the like. With this, usability is improved.

### Third Embodiment

The embodiment is not limited to the example described above. In a third embodiment, an example in which the processing circuit 110 causes the acquiring function 110c to acquire a second camera image during the execution of the second ultrasonic scan at Step S300, executing processing on the basis of the acquired image, will be explained.

At Step S300, the processing circuit 110 causes the acquiring function 110c to acquire a second camera image during the execution of the second ultrasonic scan. In other words, the processing circuit 110 causes the acquiring function 110c to acquire the second camera image resultant of capturing an image of the ultrasound probe and the subject during the time in which the second ultrasonic image data is being collected. At Step S310, the processing circuit 110 then causes the generating function 110h to generate guide information using an artificial intelligence (AI), for example, on the basis of the difference between the second camera image acquired during the collection of the second ultrasonic image data at Step S300, and the first camera image in the reference image registered at Step S120. As one example, the processing circuit 110 causes the generating function 110h to calculate the distance by which the ultrasound probe 5 is moved, and to generate guide information indicating that it is desirable to move the ultrasound probe 5 in a predetermined direction, on the basis of the second camera image captured during the collection of the second ultrasonic image data, and the first camera image captured during the collection of the first ultrasonic image data. The processing circuit 110 then causes the display control function 110d to display the generated guide information on the display 135.

As one example, the processing circuit 110 may cause the display control function 110d to display the first camera image and the second camera image in a manner superimposed over each other, and to guide the user so that the user can acquire the cross section by matching the position of the ultrasound probe 5 in the second camera image with the position of the ultrasound probe 5 in the first camera image. As another example, the processing circuit 110 may also cause the display control function 110d to display an icon indicating the position and the orientation of the ultrasound probe at the time when the first ultrasonic image data is acquired, in a manner superimposed over the second camera image, on the basis of the first position information that is information of a relative position of the ultrasound probe 5 and the subject during the time in which the first ultrasonic image data is acquired.

As described above, in the third embodiment, a camera image is collected while the second ultrasonic scan is being executed, and the guide information is automatically generated on the basis of the camera image. With this, usability is improved.

### OTHER EMBODIMENTS

The embodiment is not limited to thereto. For example, at Step S230, the processing circuit 110 may cause the acquiring function 110c to use video data before and after the first ultrasonic scan, as the information for enabling the user to acquire the cross section determined by the determining unit. In other words, an ultrasonic diagnostic apparatus 10 according to a fourth embodiment can present the procedures for scanning the ultrasound probe 5 to the user, using video data. The video data may be edited data resultant of deleting the data not necessary for guiding the ultrasound probe 5, for example, instead of unedited video data.

Furthermore, as another embodiment, although the example described in the first embodiment is an example in which the storage device 132 is included in the medical image processing apparatus 100, the embodiment is not limited thereto, and the storage device 132 may be configured as a cloud database, and the ultrasonic diagnostic apparatus 10 may refer to the information retained on the cloud database, as appropriate. An example of the advantage of using a cloud database for the storage device 132 is the ease of updating and reflecting information. By using a cloud as the storage device 132, the information for enabling the user to acquire the cross section determined at Step S220 by the determining function 110g, the information being acquired at Step S230, may be information stored in the cloud and enabled to be updated on the basis of a diagnosis guideline.

Furthermore, as another embodiment, when the reference image registered at Step S120 is an image acquired without retaining any information of the probe position and angle, the processing circuit 110 may cause the determining function 110g to automatically determine which cross section the reference image corresponds to, at Step S220. If the database stored in the storage device 132 already has the information of the position and angle of the probe corresponding to the same cross section as the determined cross section, such information may be given to the protocol. In other words, the processing circuit 110 causes the determining function 110g to add such information to the examination procedure information having been already registered, as the examination procedure information.

As still another embodiment, because an appropriate probe position and angle differs depending on the body shape of the patient, the processing circuit 110 may cause the registering function 110e to retain the information of the position or the angle of the ultrasound probe 5 at the time when the reference image is acquired using the position sensor, the pressure sensor, or a camera image, give such information to the protocol, and add such information to the examination procedure information having been already registered, at Step S120.

According to at least one of the embodiments described above, it is possible to improve the usability.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising a processing circuit configured:
to determine which cross section first ultrasonic image data represents, the first ultrasonic image data being image data designated as a reference image for an examination including a process of acquiring an ultrasonic image;
to acquire information for enabling a user to acquire the determined cross section; and
to store the acquired information in a storage device in association with the first ultrasonic image data.

2. The ultrasonic diagnostic apparatus according to claim 1, wherein the information includes at least one of a scan position for acquiring the cross section, an ultrasound probe scan policy, and an anatomical schema.

3. The ultrasonic diagnostic apparatus according to claim 1, wherein the processing circuit is configured to determine the cross section based on a trained model having been trained with labeled data.

4. The ultrasonic diagnostic apparatus according to claim 1, wherein
the storage device is a cloud, and
the information is information that is stored on the cloud and that is updatable based on an update of a diagnosis guideline.

5. The ultrasonic diagnostic apparatus according to claim 1, wherein the processing circuit is configured to acquire the information using information obtained from a position sensor or a pressure sensor, or using a camera image, at time when a first ultrasonic scan for acquiring the first ultrasonic image data is executed.

6. The ultrasonic diagnostic apparatus according to claim 1, wherein
the processing circuit is configured to, upon receiving an execution of the examination that is registered, display a plurality of examination procedures as a list, in an order of execution, on a display, and
the processing circuit is configured to, when the examination procedure is to be executed, acquire the information associated with the first ultrasonic image data from the storage device, and to display the information on the display.

7. The ultrasonic diagnostic apparatus according to claim 6, wherein the processing circuit is configured to display second ultrasonic image data acquired during an execution of the examination procedure, on the display.

8. The ultrasonic diagnostic apparatus according to claim 1, wherein the processing circuit is configured to acquire a first camera image resultant of capturing an image of an ultrasound probe position and a subject at time when the first ultrasonic image data is acquired, as the information.

9. The ultrasonic diagnostic apparatus according to claim 8, wherein the processing circuit is configured:
to acquire second position information that is a relative position of the ultrasound probe and the subject at time when the second ultrasonic image data is being collected; and
to display an icon indicating a current position and an orientation of the ultrasound probe, in a manner superimposed over the first camera image based on the second position information.

10. The ultrasonic diagnostic apparatus according to claim 1, wherein the processing circuit is configured to acquire first position information that is a relative position of an ultrasound probe and a subject at time when the first ultrasonic image data is acquired, as the information.

11. The ultrasonic diagnostic apparatus according to claim 10, wherein the processing circuit is configured:
to acquire a second camera image resultant of capturing an image of the ultrasound probe and the subject at time when the second ultrasonic image data is being collected; and
to display an icon indicating a position and an orientation of the ultrasound probe at time when the first ultrasonic image data is acquired, in a manner superimposed over the second camera image, based on the first position information.

12. The ultrasonic diagnostic apparatus according to claim 5, wherein the processing circuit is configured to acquire video data before and after the first ultrasonic scan, as the information.

13. The ultrasonic diagnostic apparatus according to claim 12, wherein the video data is edited data resultant of deleting data unnecessary for guiding the ultrasound probe.

14. An ultrasonic diagnostic apparatus comprising a processing circuit configured to, upon receiving an execution of a registered examination procedure for an examination including a process of acquiring an ultrasonic image, display the examination procedure as a list in an order of execution, on a display, wherein
the processing circuit is configured to, when the examination procedure is to be executed, acquire information associated with first ultrasonic image data, the information being information for enabling a user to acquire a cross section of the first ultrasonic image data that is designated as a reference image for the examination, from a storage device, and to display the information on the display.

15. An ultrasonic diagnostic method comprising:
determining which cross section first ultrasonic image data represents, the first ultrasonic image data being image data designated as a reference image for an examination including a process of acquiring an ultrasonic image;
acquiring information for enabling a user to acquire the determined cross section; and
storing the acquired information in a storage device in association with the first ultrasonic image data.
